(19) United States
(12) Patent Application Publication
(10) Pub. No.: US 2024/0344950 A1
(43) Pub. Date: Oct. 17, 2024

(54) ODOR MEASURING DEVICE

(71) Applicant: TAIYO YUDEN CO., LTD., Tokyo (JP)

(72) Inventor: Yosuke ONDA, Tokyo (JP)

(73) Assignee: TAIYO YUDEN CO., LTD., Tokyo (JP)

(21) Appl. No.: 18/750,375

(22) Filed: Jun. 21, 2024

Related U.S. Application Data
(63) Continuation of application No. PCT/JP2022/039671, filed on Oct. 25, 2022.

(30) Foreign Application Priority Data
Dec. 24, 2021 (JP) 2021-211173

(51) Int. Cl. G01N 5/02; G01N 33/00

(52) U.S. Cl. CPC G01N 5/02; G01N 33/0031

(57) ABSTRACT

An odor measuring device includes a sensor substrate having a first main surface having a flow path region constituting a flow path, the flow path region including a first region and a second region, an odor sensor that is mounted on the first region and detects an odorous substance, and a first protective layer provided on the second region.

### CROSS-REFERENCE TO RELATED APPLICATION

This application is a continuation application of International Application No. PCT/JP2022/039671 filed on Oct. 25, 2022, which claims priority to Japanese Patent Application No. 2021-211173 filed on Dec. 24, 2021, the entire contents of which are incorporated herein by reference.

### FIELD

A certain aspect of the present disclosure relates to an odor measuring device for measuring an odor.

### BACKGROUND

Some odor sensors measure an odorous substance using a frequency variation caused by mass addition of a piezoelectric resonator such as a quartz crystal microbalance (QCM), a surface acoustic wave (SAW) resonator, or a film bulk acoustic resonator (FBAR), or a resistance value change caused by gas combustion on a surface of an oxide semiconductor material. In addition, research has been conducted in recent years on a technique for identifying the type of odor by patterns using a plurality of odor sensors that adsorb different odorous substances.

In the field of gas analysis such as odor measurement, detection accuracy can be improved by preventing adhesion of a detection target substance to a portion other than a sensor. For example, Japanese Patent Application Laid-Open No. 2008-232799 (Patent Document 1) discloses a pyrolysis gas chromatograph device in which the inner surface of a flow path is coated. Further, Japanese Patent Application Laid-Open No. 2009-128054 (Patent Document 2) discloses a gas collecting device including an air intake tube made of a fluorinated resin to which gas is less likely to adhere.

Further, Japanese Patent Application Laid-Open No. 2012-194088 (Patent Document 3) discloses a gas sampling and measuring device in which the inner wall surface of a sampling container is coated with high-density fused silica or the like to reduce the generation of outgas and the adhesion of gas components. As an electrode structure for liquid analysis, Japanese Patent Application Laid-Open No. 2019-90709 (Patent Document 4) discloses an electrode structure including an electrode provided on a base material and a protective layer made of amorphous carbon or the like and formed on the electrode.

### SUMMARY

Here, a peripheral circuit is essential for operating the odor sensor, and the peripheral circuit is often mounted on a circuit board. However, the adhesion of odorous substances to the circuit board causes a reduction in sensitivity, a drift in properties and/or a reduction in measurement reproducibility.

When detecting an odor in a low concentration region, it is necessary to detect a weaker electric signal or high-frequency signal, and the peripheral circuit is also increased in size and complicated, and therefore, adhesion of the odorous substance to the circuit board becomes remarkable.

Even when coating is performed on a circuit board, the coating as described in Patent Documents 1 to 4 is performed by a dry film forming process such as chemical vapor deposition (CVD) or sputtering. Such coatings often involve bias application or substrate heating during the process, which can cause failure of mounted electronic components or poor contact of reflow-mounted components.

In addition, the circuit board is often a printed circuit board made of an organic material such as FR4 (Flame Retardant Type 4), which causes outgassing from the organic material and impairs the adhesion between the coating and the circuit substrate.

In view of the above circumstances, an object of the present disclosure is to provide an odor measuring device capable of inhibiting adhesion of an odorous substance to a circuit board.

In one aspect of the present disclosure, there is provided an odor measuring device including: a sensor substrate having a first main surface having a flow path region constituting a flow path, the flow path region including a first region and a second region; an odor sensor that is mounted on the first region and detects an odorous substance; and a first protective layer provided on the second region.

In another aspect of the present disclosure, there is provided a sensor substrate that has a first main surface and a second main surface, the first main surface having a first region and a second region, the first region including a sensor region to which an odor sensor for detecting an odorous substance is bonded and an electrode region that is adjacent to the sensor region and provided with an electrode to which the odor sensor is connected, the second main surface being a main surface opposite to the first main surface; a first conductive pattern that is provided on the second main surface and is electrically connected to the electrode; a metal layer that is provided in the second region; a first protective layer that is stacked on the metal layer and inhibits adhesion of an odorous substance; an odor sensor mounted on the first region and electrically connected to the electrode; an electronic component mounted on the second main surface and electrically connected to the first conductive pattern; and a sensor chamber having the first main surface as an inner surface.

In another aspect of the present disclosure, there is provided an odor measuring device including: a sensor substrate having a first main surface including a flow path region constituting a flow path, the sensor substrate including a first protective layer provided in the flow path region, the first protective layer inhibiting adhesion of an odorous substance contained in a gas flowing through the flow path; and an odor sensor that is mounted in the odor sensor mounting region and detects an odorous substance contained in the gas.

In another aspect of the present disclosure, there is provided an odor measuring device including: a printed circuit board having a front surface having an arrangement region where an odor sensor is arranged and a region serving as a region of a gas flow path surrounding the arrangement region of the odor sensor, and a back surface that faces the front surface and is provided with a conductive pattern connected to an electrode provided in the arrangement region of the odor sensor; an odor sensor electrically connected to the electrode in the arrangement region and fixed; a layer made of Cu and provided on the printed circuit board corresponding to an entire region of the gas flow path except for the arrangement region; a protective layer provided on the layer made of Cu via an intermediate layer; a housing that forms a space of a sensor chamber in combination with the printed circuit board that is an outer periphery of a region of the gas flow path; and a circuit element that is connected to the conductive pattern on the back surface and constitutes a drive circuit for driving the odor sensor.

### BRIEF DESCRIPTION OF THE DRAWINGS

FIG. 1 is a perspective view of an odor measuring device in accordance with an embodiment;

FIG. 2 is an exploded perspective view of the odor measuring device;

FIG. 3 is a sectional view of the odor measuring device;

FIG. 4 is a cross-sectional view of a sensor substrate, an odor sensor, and electronic components included in the odor measuring device;

FIG. 5 is a schematic view illustrating a flow path in the odor measuring device;

FIG. 6 is a schematic view illustrating a flow path region and a non-flow path region on a first main surface of the sensor substrate included in the odor measuring device;

FIG. 7 is a schematic view illustrating the flow path region and the non-flow path region on the first main surface of the sensor substrate;

FIG. 8 is a cross-sectional view of the sensor substrate included in the odor measuring device;

FIG. 9 illustrates the sensor substrate, the odor sensor, and electronic components included in the odor measuring device;

FIG. 10 is a cross-sectional view illustrating a mounting structure of the odor sensor by wire bonding on the sensor substrate of the odor measuring device;

FIG. 11 is a cross-sectional view illustrating a mounting structure of the odor sensor by solder reflow on the sensor substrate of the odor measuring device;

FIG. 12 is a cross-sectional view illustrating a shield member bonded to the sensor substrate of the odor measuring device;

FIG. 13 illustrates a second protective layer provided in the odor measuring device;

FIG. 14 illustrates a reference element provided in the odor measuring device;

FIG. 15 illustrates the reference element provided in the odor measuring device;

FIG. 16 is a schematic view illustrating a method of manufacturing the odor measuring device;

FIG. 17 is a schematic view illustrating the method of manufacturing the odor measuring device;

FIG. 18 is a schematic view illustrating the method of manufacturing the odor measuring device;

FIG. 19 is a cross-sectional view illustrating the electronic components mounted on the first main surface of the sensor substrate of the odor measuring device;

FIG. 20 is a cross-sectional view of the sensor substrate including a support member, which is included in the odor measuring device;

FIG. 21 is a schematic view illustrating a method of manufacturing the sensor substrate including the support member, which is included in the odor measuring device;

FIG. 22 is a schematic view illustrating an arrangement of the flow path region of the sensor substrate of the odor measuring device;

FIG. 23 is a schematic view illustrating an arrangement of the flow path region of the sensor substrate of the odor measuring device;

FIG. 24 is a schematic view illustrating an arrangement of the flow path region of the sensor substrate of the odor measuring device;

FIG. 25 is a cross-sectional view of the sensor substrate on which a plurality of odor sensors are mounted, which is provided in the odor measuring device;

FIG. 26 is a plan view of the sensor substrate on which the odor sensors are mounted, which is provided in the odor measuring device;

FIG. 27 is a cross-sectional view of a plurality of sensor substrates connected to the support substrate, which are provided in the odor measuring device;

FIG. 28 is a plan view of the sensor substrates connected to the support substrate, which are provided in the odor measuring device;

FIG. 29 is a cross-sectional view illustrating the sensor substrates connected to the support substrate and a housing member, which are included in the odor measuring device;

FIG. 30 is a cross-sectional view of sensor substrates that are connected to the support substrate and have tapered end surfaces, which are included in the odor measuring device;

FIG. 31 is an enlarged view of FIG. 30;

FIG. 32 is a schematic view illustrating a method of manufacturing the sensor substrate having a tapered end surface;

FIG. 33 is a cross-sectional view illustrating a through hole formed in a substrate body of the sensor substrate of the odor measuring device;

FIG. 34 is a cross-sectional view illustrating a plug for filling the through hole;

FIG. 35 is a cross-sectional view illustrating a through hole and a first protective layer provided on the substrate body of the sensor substrate of the odor measuring device;

FIG. 36 is a cross-sectional view illustrating a wiring structure of the sensor substrate of the odor measuring device;

FIG. 37 is a cross-sectional view illustrating the wiring structure of the sensor substrate and a cover provided in the odor measuring device;

FIG. 38 is a cross-sectional view illustrating a flip-chip mounted odor sensor and a flow path provided in the odor measuring device; and

FIG. 39 is a schematic view illustrating the flip-chip mounted odor sensor and the flow path provided in the odor measuring device.

### DETAILED DESCRIPTION

Hereinafter, an odor measuring device in accordance with an embodiment of the present disclosure will be described with reference to the drawings. In the following description, the term “odor” refers to an aggregate of a plurality of types of odorous substances. Examples of odorous substances include molecules such as acetone and toluene.

The adsorption film of each odor sensor described later has selectivity of an odorous substance it adsorbs. The respective adsorption films of the odor sensors adsorb different types of odors. In other words, the adsorption films of the odor sensors adsorb different types of odorous substances and different amounts of the odorous substances.

In the present embodiment, the type of odor, which is an aggregate of the odorous substances, is determined by comprehensively determining the amounts of the odorous substances measured by the respective odor sensors. The types of odors include fruit odors, body odors, burnt odors caused by breaking of power cords, and odors of toxic drugs prohibited by law. The details will be described below.

### [Configuration of Odor Measuring Device]

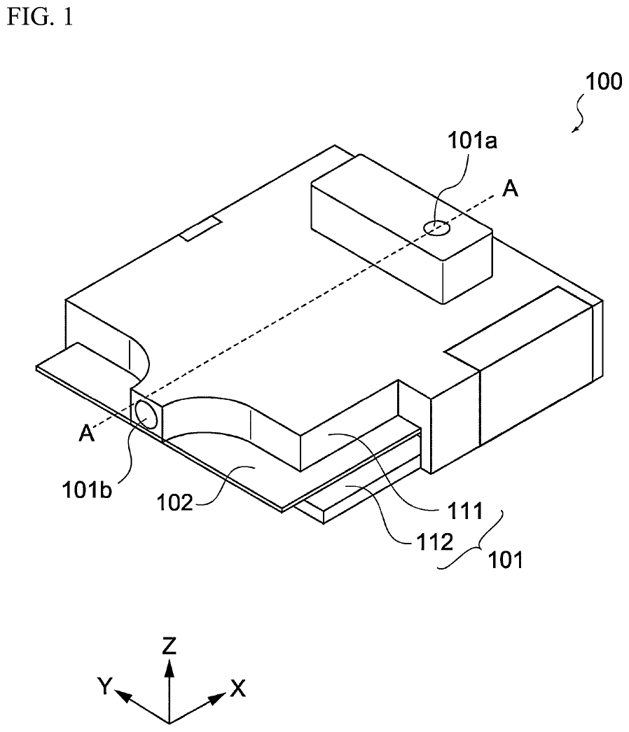

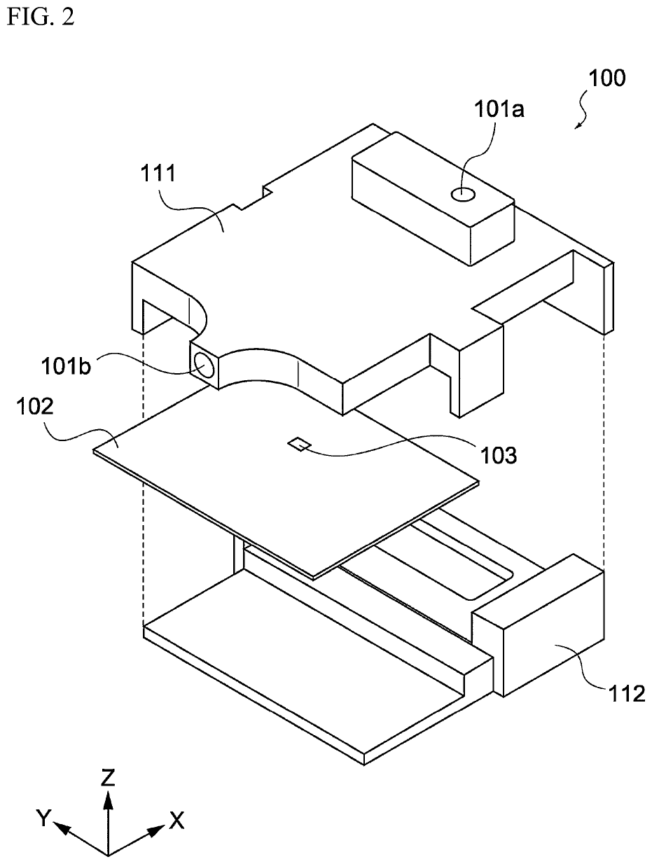

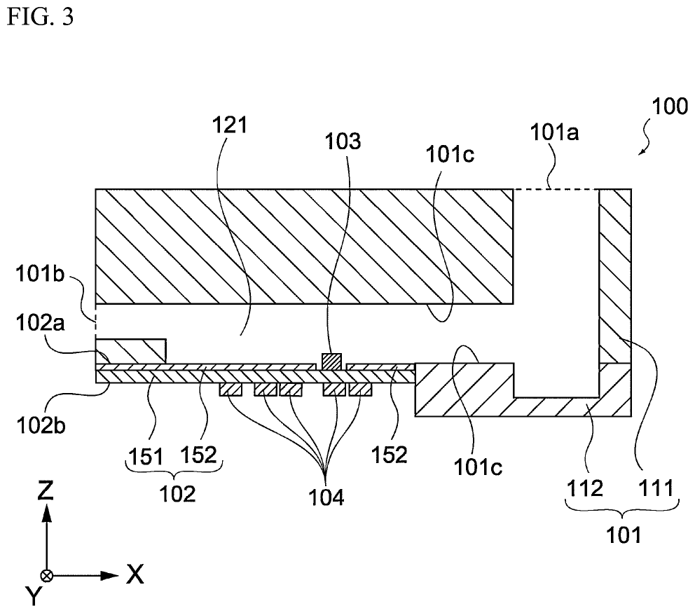

FIG. 1 is a perspective view of an odor measuring device 100 in accordance with a present embodiment, and FIG. 2 is an exploded perspective view of the odor measuring device 100. FIG. 3 is a cross-sectional view of the odor measuring device 100, taken along line A-A in FIG. 1. As illustrated in FIG. 1 to FIG. 3, the odor measuring device 100 includes a housing member 101, a sensor substrate 102, an odor sensor 103, and electronic components 104.

The housing member 101 is joined to or combined with the sensor substrate 102 to form a gas flow path. Hereinafter, the gas flowing through the flow path is referred to as a measurement target gas.

The housing member 101 may be composed of two parts, a first housing member 111 and a second housing member 112, as illustrated in FIG. 2. The first housing member 111 and the second housing member 112 are joined to each other with the sensor substrate 102 interposed therebetween. The first housing member 111 and the second housing member 112 can be joined by screwing, an adhesive, or metal-to-metal welding. As illustrated in FIG. 1, the housing member 101 is provided with an intake port 101a and an exhaust port 101b that are openings opened to the outside of the housing.

As illustrated in FIG. 3, an upper plate having an opening for fitting the sensor substrate 102 may be provided on the surface of the second housing member 112, and the sensor substrate 102 may be combined with the opening. Whether the sensor substrate 102 is joined or fitted to the housing, the sensor substrate 102 forms at least a part of the bottom of the flow path.

As illustrated in FIG. 3, the inner surface of the housing member 101 is referred to as an inner surface 101c. The housing member 101 is made of a heat-resistant resin such as polytetrafluoroethylene (PTFE) or a metal such as aluminum or stainless steel.

For example, the intake port 101a is located on the upper surface of the first housing member 111, from which a cylindrical flow path extends downward. The structure is illustrated on the right side of FIG. 3. At the bottom or in the middle of the cylindrical flow path, there is an inlet port connected to a sensor chamber 121, and the inlet port communicates with the sensor chamber 121. The exhaust port 101b is provided on the inside wall opposed to the inlet port through a horizontally or upwardly directed flow path.

The housing member 101 has a cavity defined by the upper surface of the inner wall and the inner wall surface extending downward from the periphery of the upper surface, and the portion corresponding to the bottom surface is open. In this embodiment, the housing member 101 is formed by molding in a die.

The sensor substrate 102 described later is brought into contact with this open portion to form the bottom portion, thereby forming the space of the sensor chamber 121. The sensor chamber 121 is provided with the odor sensor 103 alone at the sensor chamber 121 side. Alternatively, as few elements as possible are placed with the odor sensor 103 and a limited number of elements, e.g., ICs that need to be placed in close proximity, and the rest are arranged on the back surface of the sensor substrate 102.

The sensor chamber 121 preferably has a substantially rectangular parallelepiped shape, a dome shape, or the like.

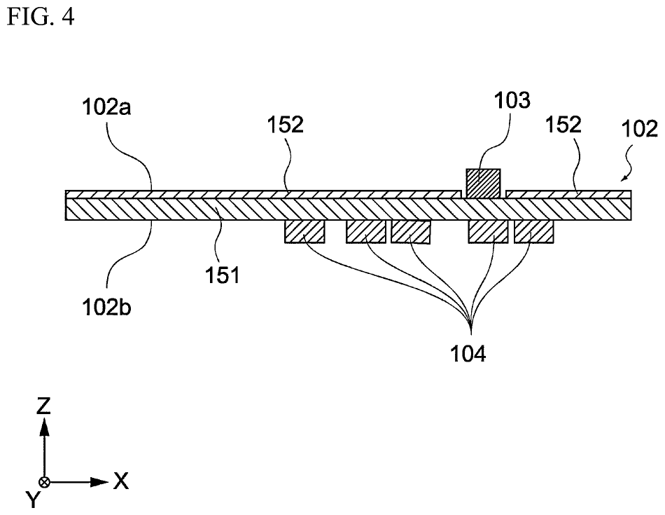

The sensor substrate 102 is a substrate on which the odor sensor 103 and the electronic components 104 are mounted. FIG. 4 is a cross-sectional view of the sensor substrate 102, the odor sensor 103, and the electronic components 104. As illustrated in FIG. 4, the sensor substrate 102 includes a substrate body 151 and a first protective layer 152, and has a first main surface 102a and a second main surface 102b. The first main surface 102a and the second main surface 102b are main surfaces opposite to each other.

The sensor substrate 102 is formed of a printed circuit board. For example, a ceramic substrate may be used, but a printed circuit board is used here in consideration of damage prevention. Hereinafter, the sensor substrate 102 may be referred to as a printed circuit board, the first main surface 102a may be referred to as the front surface of the printed circuit board, and the second main surface 102b may be referred to as the back surface of the printed circuit board.

The sensor substrate 102 may be formed of a multilayer substrate because elements are also mounted on the back surface. If the sensor substrate 102 is FR4, there is a conductive pattern between layers of epoxy resin, and the resin surfaces are exposed on both the front and back surfaces.

When only the odor sensor 103 is mounted on the front surface of the sensor substrate 102, the connection electrodes of the odor sensor 103 are exposed, and when only the odor sensor 103 and the IC are mounted on the front surface of the sensor substrate 102, the electrodes required for the connection are exposed. On the back surface, a conductive pattern is formed via through holes or vias, and other electronic components 104 for driving the sensor are mounted.

In addition, solder resists may be used on both surfaces of the printed circuit board. In this case, the conductive pattern including wiring lines and electrodes may be covered with the solder resist, except for the connection electrodes. The first protective layer 152 may be provided so as to cover the resist layer.

The description “the connection electrode is exposed” is made because the first protective layer 152 is provided on the outermost surface of the printed circuit board. The first protective layer 152 is provided to prevent adsorption and desorption of the odorous substance to and from a printed board material, a solder resist, or a conductive pattern exposed in the sensor chamber 121.

In the odor measuring device 100, as illustrated in FIG. 3, a space is surrounded by the inner surface 101c and the sensor substrate 102 to form the sensor chamber 121. The first main surface 102a is a main surface at the sensor chamber 121 side, and the second main surface 102b is a main surface at the opposite side to the sensor chamber 121.

As described above, the odor sensor 103 is selected in consideration of mountability on the first main surface 102a. Here, a QCM (Quartz Crystal Microbalance) sensor is selected as the odor sensor 103, but a rectangular parallelepiped chip type is also effective in consideration of mountability.

The chip type generally has a sensitive film on the front surface, and the back surface can be fixed by soldering or an adhesive. The chip surface is provided in parallel with the direction of the flow of the odor taken in from the inlet port. This is because the odor is easily detected. The chip type sensor is an FBAR, a MEMS type sensor having a resistance element or a vibration element on the surface thereof, an optical sensor, an electrochemical sensor, a thermistor type sensor, or the like.

Here, the QCM includes a vibrator and an adsorption film covering the surface of the vibrator. The vibrator vibrates at a constant resonance frequency when a voltage is applied. The resonance frequency is, for example, 9 MHz. The adsorption film adsorbs a specific odorous substance. When the vibrator is vibrated at a constant resonance frequency, adsorption of the odorous substance on the adsorption film increases the weight of the adsorption film and decreases the resonance frequency of the vibrator. When the odorous substance adsorbed to the adsorption film is desorbed, the weight of the adsorption film decreases, and the resonance frequency of the vibrator increases. The odor sensor 103 outputs the amount of variation in the resonance frequency as a detection value.

A plurality of the odor sensors 103 may be mounted on the first main surface 102a. In this case, the adsorption film of each odor sensor is made of a material different for each odor sensor 103. The odor contained in the measurement target gas contains one or more odorous substances. By using different adsorption films for the odor sensors 103, respectively, a plurality of types of odorous substances can be detected. The material used for the adsorption film is appropriately selected according to the type of odor to be measured.

Specifically, cellulose, a fluorine-based polymer, lecithin, a phthalocyanine compound, a porphyrin compound, polyimide, polypyrrole, polystyrene, an acrylic polymer, sphingomyelin, polybutadiene, polyisoprene, a polyvinyl alcohol-based polymer, or an organic metal structure (MOF) such as UiO-66, MIL-125, or ZIF-8 can be used as the adsorption film. The adsorption film may include any one of these materials or a combination of two or more of these materials.

The odor sensor 103 may be any sensor capable of detecting adsorption of an odorous substance, and may be a polymer or ceramic-based resistance-type odor sensor, a capacitance-type odor sensor in which a dielectric is interposed between two electrodes, or a resonator-type odor sensor using a film bulk acoustic resonator (FBAR), a surface acoustic wave (SAW) resonator, or the like, in addition to the QCM of the present embodiment.

In FIG. 3, only the odor sensor 103 is provided on the first main surface 102a, and the electronic components 104 are mounted on the second main surface 102b and electrically connected to the odor sensor 103. The electronic components 104 may be mounted in any location other than the first main surface 102a of the sensor substrate 102, in addition to the second main surface 102b. The electronic components 104 are control circuit elements of the odor sensor 103, supplies a drive signal to the odor sensor 103, acquires a detection value of the odor sensor 103, and executes signal processing. When the odor sensor 103 is a resonator-type odor sensor, the electronic components 104 may include an oscillation circuit and a frequency counter circuit. The electronic components 104 may be a discrete component or an integrated circuit (IC). The number of the electronic components 104 may be one or more.

As described above, the resonator-type odor sensor 103 requires an oscillation circuit. In order to achieve good oscillation, the oscillation circuit and the odor sensor 103 need to be connected by a short path, and in this sense, only the odor sensor 103 and an IC with a built-in oscillation circuit may be mounted on the front surface of the printed circuit board. In this manner, since the number of components mounted on the front surface of the printed circuit board can be minimized, the formation area of the first protective layer 152 in the flow path can be maximized, and the generation of outgas can be further reduced. In this case, the frequency counter circuit may not be necessarily provided on the front surface of the printed circuit board as long as the frequency counter circuit is electrically connected to the resonator-type odor sensor 103.

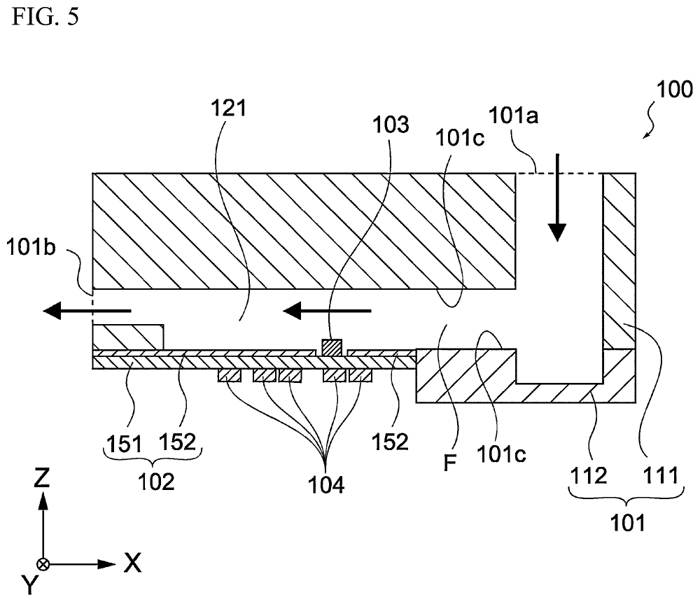

In the odor measuring device 100, the odor sensor 103 detects an odorous substance contained in the measurement target gas. In FIG. 5, the flow of the measurement target gas in the odor measuring device 100 is indicated by arrows. As illustrated in FIG. 5, in the odor measuring device 100, the measurement target gas flows into the sensor chamber 121 from the intake port 101a, flows through the sensor chamber 121, and is discharged from the exhaust port 101b. The sensor chamber 121 is a chamber in which the odor sensor 103 is installed and is also a flow path of gas.

The odor measuring device 100 includes a gas delivery mechanism such as a pump or a fan (not illustrated), and the gas delivery mechanism causes the measurement target gas to flow from the intake port 101a to the exhaust port 101b. Alternatively, the measurement target gas may be pumped from a pressurized vessel such as a cylinder and flow from the intake port 101a to the exhaust port 101b. Hereinafter, a path through which the measurement target gas flows in the odor measuring device 100, that is, a path from the intake port 101a to the exhaust port 101b through the sensor chamber 121 is referred to as a “flow path F”.

The first main surface 102a of the sensor substrate 102 has a “flow path region” and a “non-flow path region”. The flow path region is a region constituting the flow path F in the first main surface 102a, that is, a region forming a part of the inner surface of the sensor chamber 121. The non-flow path region is a region that does not constitute the flow path F in the first main surface 102a, that is, a region that does not form the inner surface of the sensor chamber 121.

As illustrated in FIG. 1, the sensor substrate 102 is in contact with the side wall of the opening of the first housing member 111. Therefore, as described above, the partition wall having the inner side wall of the housing member 101 is in contact with the sensor substrate 102, and the outside of the partition wall is out of the housing member 101, like the root portion of the lead line indicated by the symbol 102 in FIG. 1. Therefore, the portion of the sensor substrate 102 corresponding to the internal space serves as the sensor chamber 121 and also as a gas flow path. On the other hand, the portion of the sensor substrate 102 that is in contact with the partition wall and the outside thereof are non-flow path regions.

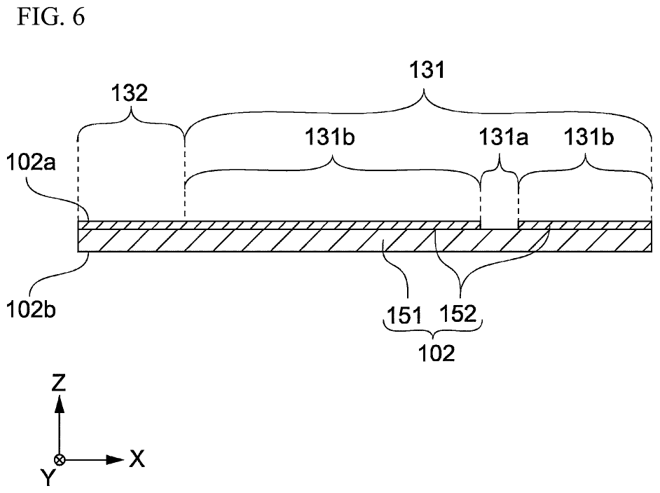

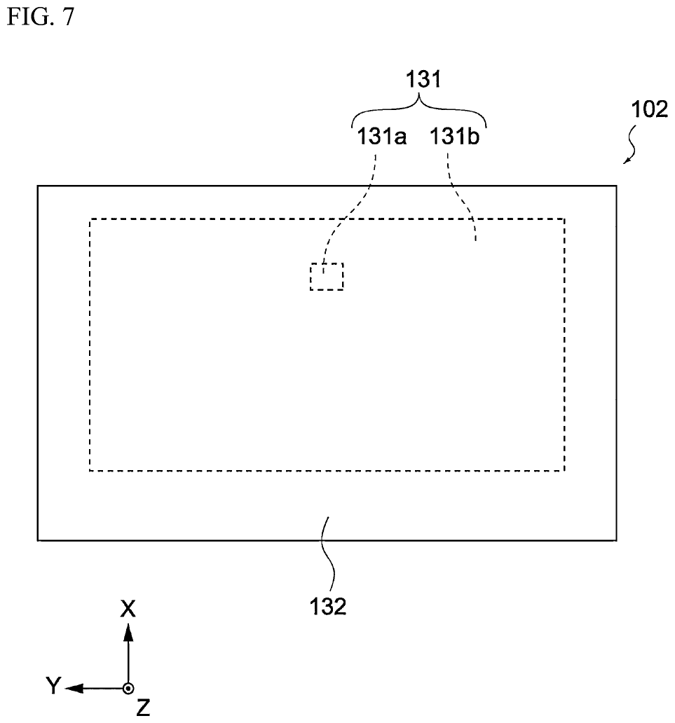

FIG. 6 is a cross-sectional view of a flow path region 131 and a non-flow path region 132, and FIG. 7 is a plan view of the flow path region 131 and the non-flow path region 132.

The non-flow path region 132 is a region of the first main surface 102a to which the housing member 101 (partition wall) is joined as illustrated in FIG. 3, or which protrudes from the housing member 101 as illustrated in FIG. 1. The flow path region 131 is a region other than the non-flow path region 132 in the first main surface 102a.

The first main surface 102a may not necessarily include the non-flow path region 132, and the entire region may be the flow path region 131.

As illustrated in FIG. 6 and FIG. 7, the flow path region 131 is composed of an odor sensor mounting region 131a and a non-mounting region 131b. The odor sensor mounting region 131a is a region where the odor sensor 103 is mounted. The non-mounting region 131b is a region adjacent to the odor sensor mounting region 131a, and is a region where the odor sensor 103 and other electronic components are not mounted. As will be described later, the non-mounting region 131b further includes a conductive pattern hidden under the protective layer. The first protective layer 152 is formed on the uppermost layer via a solder resist, a plating layer, or the like, and the conductive pattern is a portion that is not exposed from the first protective layer 152. A plurality of the odor sensor mounting regions 131a may be provided.

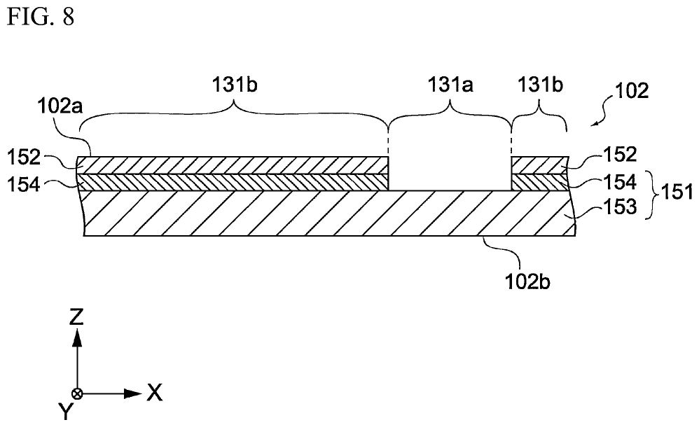

FIG. 8 is an enlarged view of the sensor substrate 102. As illustrated in FIG. 8, the sensor substrate 102 includes the substrate body 151 and the first protective layer 152. The substrate body 151 includes a base material layer 153 and a metal layer 154, and is a printed circuit board such as FR4 (Flame Retardant Type 4). The base material layer 153 is made of an insulating material, for example, a glass epoxy material. A wiring structure (not illustrated) may be provided on the base material layer 153.

An overview of the first protective layer 152 will be described. The first protective layer 152 is a film that does not affect or is unlikely to affect the adsorption and desorption characteristics of the odor sensor 103. For example, a film that does not adsorb the target odor. To be precise, “hard to do” is more accurate than “not to do”.

For example, in the case of a metal, the oxide product has a portion where various oxide particles overlap, and this portion easily forms an adsorption site, and therefore, metals that do not form oxides are preferable. Therefore, the first protective layer 152 is preferably made of a noble metal such as Au, Pt, or Ag. Further, amorphous Si, DLC, artificial diamond coating, or the like may be used. This is presumably because the surface is glassy and smooth, and thus the formation of adsorption sites is suppressed. In other words, the film may be a film having a water repellent or oil repellent surface.

The first protective layer 152 is formed on the uppermost layer of the sensor substrate 102. Therefore, the degree of adhesion to the base layer is considered. No other layers need to be interposed between the base layer and the first protective layer 152, as long as they can adhere to each other. However, a film called an intermediate layer or a buffer layer is usually employed in consideration of adhesion to the base layer.

As an example of the present embodiment, an epoxy substrate is adopted as the sensor substrate 102. This substrate is characterized in that it is finally both a substrate having a protective film and a circuit board. That is, since a printed circuit board often uses a noble metal, for example, Au on Cu, the process and structure thereof may be adopted. For example, Au is used on the outermost surface as an electrode for soldering the printed circuit board or an electrode for wire bonding. In general, a conductive pattern made of Cu is formed on the intermediate layer or the outermost surface of the printed circuit board by plating or bonding of Cu foils. Copper is used in consideration of resistance and cost. The conductive patterns on the uppermost layers of the front and back surfaces of the printed circuit board are coated with a solder resist, and only the connection portions are exposed from the resist film. The exposed portion is plated with Cu, Ni, and Au, or Ni and Au in this order by partial plating or the like. There are many other multilayering methods and electrode materials, but details thereof are omitted.

A method of manufacturing a printed circuit board will be described below. First, there is a process of forming a conductive pattern. At least one layer of conductive pattern is formed on each of the front and back surfaces, and the front and back surfaces are connected to each other by through holes or the like.

### (Number of Layers of Printed Circuit Board)

As described above, the odor sensor 103 alone or the odor sensor 103 and the IC element (which may be bare or sealed) are mounted on the front surface of the printed circuit board, and various electronic components are mounted on the conductive pattern as the drive circuit of the odor sensor 103 on the second main surface 102b. The printed circuit board is therefore a multilayer board with at least two layers.

### (Conductive Pattern and Electrode Structure of Printed Circuit Board)

(Here, the conductive pattern is formed of an electrode, a wiring line, a wiring line integrated with an electrode, or the like).

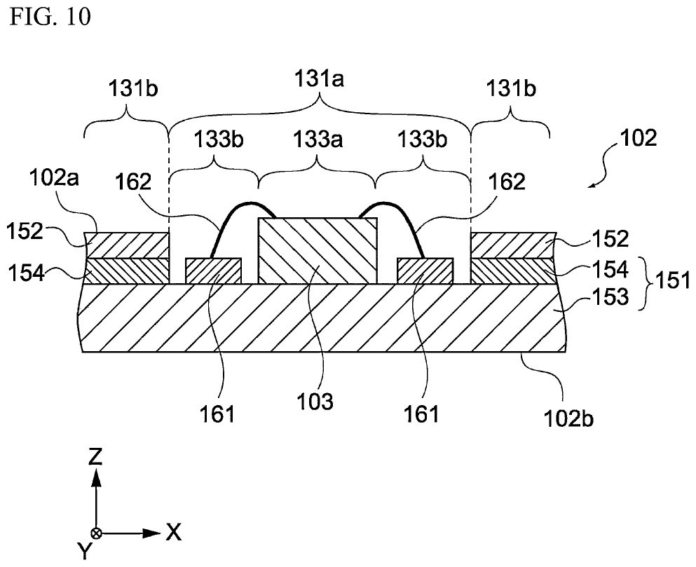

Electrodes 161 (see FIG. 10 described later) necessary for mounting the odor sensor 103 are provided on the front surface of the printed circuit board, and leads are soldered to the two electrodes 161 in the case of a quartz crystal resonator. Elements such as MEMS (Micro Electro Mechanical Systems) sensors and FBARs are mainly connected by wire bonding or soldering in the case of surface mounting. In the case of wire bonding, a wire 162 (see FIG. 10 described later) connects the electrode of the odor sensor 103 and the electrode 161 of the printed circuit board. Therefore, the electrodes 161 connected to the odor sensor 103 are provided on the front surface of the printed circuit board.

The electrode 161 is formed in an island shape and is connected to a wiring line on the back surface via a through hole or a via directly below the electrode 161. In this manner, the wiring line is not exposed.

Further, a wiring line that is integrated with the electrode 161 may be extended. The first protective layer 152 having an insulating property may be formed on the wiring line except for a few wiring lines. When a wiring line is formed, the wiring line is preferably covered with a solder resist. If there is a wiring line in a portion other than the arrangement region of the sensor, the wiring line may be covered with a solder resist, and the first protective layer 152 may be formed on the solder resist.

On the back surface of the printed circuit board, elements of the driving circuit, for example, a chip device, a semiconductor device, or the like are mounted, and a conductive pattern for connecting the circuit elements is provided. Since the back surface does not constitute the inner surface of the flow path or the inner surface of the sensor chamber 121, the uppermost layer is covered with a solder resist.

### (Advantages Until First Protective Layer is Formed)

A description will be given with reference to FIG. 7. FIG. 7 illustrates the odor sensor mounting region 131a where the odor sensor 103 is mounted and the non-mounting region 131b which is a covered area with the first protective layer 152 on the front surface of the printed circuit board. The odor sensor mounting region 131a is a region where the odor sensor 103 is mounted and a region where electrodes connected to the odor sensor 103 are provided, and the enclosed portion is rectangular here. However, if the first protective layer 152 that is conductive is formed adjacent to the outside from the rectangle, short circuit occurs. Therefore, the necessary electrical separation distance is provided from this region, and a slightly wider rectangle is used as the odor sensor mounting region 131a.

The region between the dotted line indicating the odor sensor mounting region 131a and the dotted line indicating the non-mounting region 131b is the region covered with the first protective layer 152. When forming Au, which is one of the first protective layer 152, the Cu pattern on the outermost surface is used. The outermost surface of the back surface of the printed circuit board is formed of a Cu conductive pattern. At the same time as this formation, the entire non-mounting region 131b and Cu as electrodes in the odor sensor mounting region 131a are formed.

The first protective layer 152 is preferably made of the same material as the noble metal provided on the surface of the electrode. Ni and Au are applied in this order to the Cu electrode, and therefore, Ni and Au may be formed over the entire region of the non-mounting region 131b.

On the other hand, when an electrode is formed on a printed circuit board, an epoxy resin that is a substrate material is generally exposed around the electrode. The printed circuit board is covered with a solder resist except for the electrodes. Therefore, the first protective layer 152 is formed on either of the resin layers. In the case of amorphous Si or DLC, a binder may be necessary, and therefore, a binder may be formed on the resin layer, and the first protective layer 152 may be formed thereon.

The following description will be made from another viewpoint.

The metal layer 154 is provided on the base material layer 153 corresponding to the non-mounting region 131b, and is a layer that improves the adhesion between the first protective layer 152 and the base material layer 153. The metal layer 154 may be provided on the base material layer 153 in the odor sensor mounting region 131a and the non-flow path region 132. The metal layer 154 and the protective film of Au may be a multilayer film of Cu/Ni/Au in which Cu, Ni, and Au are stacked in this order or a multilayer film of Cu/Ni/Pd/Au in which Cu, Ni, Pd, and Au are stacked in this order. The metal layer 154 in the non-mounting region 131b does not have a function as wiring lines because the multilayer film is a solid film on the entire surface. However, a conductive pattern insulated by an insulating layer may be provided under the solid film. Further, as described later, the solid film may be used as a ground wiring line or a shield by being provided on the ground.

The first protective layer 152 is a layer that is provided on the metal layer 154 in the non-mounting region 131b and suppresses adhesion of odorous substances to the non-mounting region 131b. Specifically, the first protective layer 152 reduces the surface energy of the sensor substrate 102 and reduces the specific surface area by planarization, thereby reducing the amount of adhesion of the odorous substance. As illustrated in FIG. 6, the first protective layer 152 may be provided on the metal layer 154 in the non-flow path region 132. The first protective layer 152 is made of amorphous silicon, diamond-like-carbon (DLC), noble metal, or a composite layer thereof. The thickness of the first protective layer 152 is preferably 40 μm or greater and 200 μm or less. The substrate body 151 may not necessarily include the metal layer 154, and the first protective layer 152 may be directly stacked on the base material layer 153.

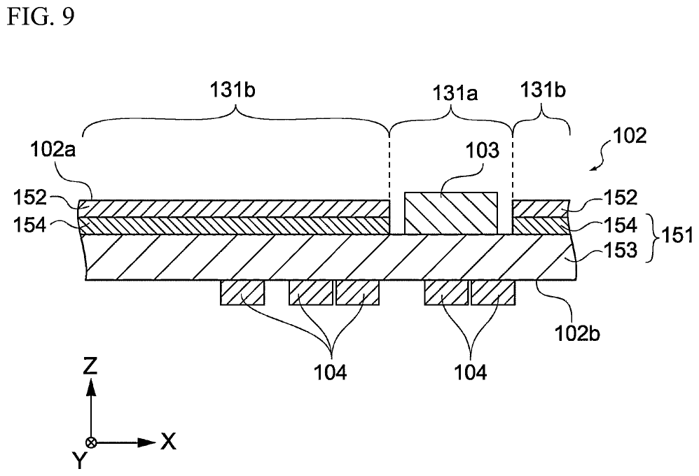

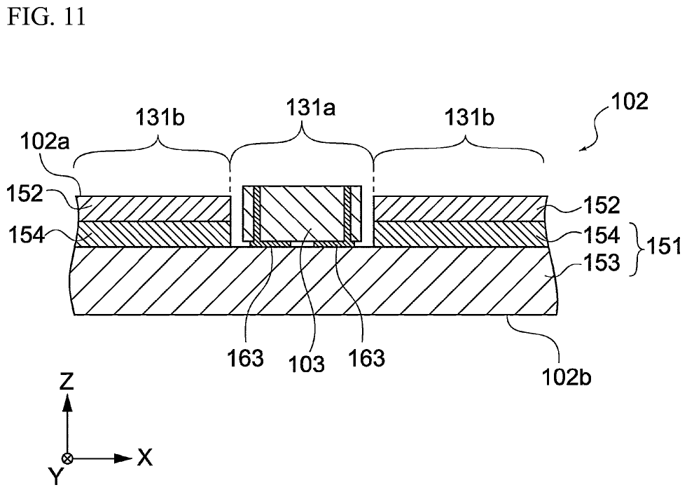

FIG. 9 is a diagram illustrating the sensor substrate 102, the odor sensor 103, and the electronic component 104. As illustrated in FIG. 9, the odor sensor 103 is mounted in the odor sensor mounting region 131a, and the electronic components 104 are mounted on the second main surface 102b. FIG. 10 and FIG. 11 are cross-sectional views illustrating a specific mounting structure of the odor sensor 103. FIG. 10 illustrates a wire-bonded type in which an FBAR or MEMS semiconductor device is employed. Further, FIG. 11 illustrates a surface mounting type in which electrodes are arranged on the back surface by TSV (Through Silicon Via Technology) or the like, which is a soldering type.

As illustrated in FIG. 10, the odor sensor 103 can be mounted by wire bonding. As illustrated in FIG. 10, the odor sensor mounting region 131a includes a sensor region 133a and an electrode region 133b, and the electrode region 133b is adjacent to the sensor region 133a. The odor sensor 103 is bonded to the sensor region 133a by a die attach material. When the chip is grounded (GND), there is an electrode for mounting, and the chip is mounted on the electrode by solder, Ag paste, or the like. In the electrode region 133b, the electrode 161 is provided apart from the metal layer 154 and electrically separated from the metal layer 154. The odor sensor 103 is electrically connected to the electrodes 161 by the wires 162. The separation distance for electrical separation between the metal layer 154 and the electrode 161 is determined by the potential difference from the chip, but is generally about 0.5 mm.

As described above, if a through hole or a via exists directly under the electrode 161, the wiring line can be eliminated, and the electrode 161 is formed in an island shape, thereby eliminating the exposure of an extra wiring layer. In addition, when the first protective layer 152 is connected to the back surface via some wiring lines for the convenience of design, the first protective layer 152 may be integrally formed on the wiring line.

As illustrated in FIG. 11, the odor sensor 103 is a surface-mounted type, and the electrodes of the sensor are provided on the back surface of the chip. In this case, solder reflow is generally used for the mounting. As illustrated in FIG. 11, the odor sensor 103 is electrically connected to the electrodes provided on the base material layer 153 by solder 163. Electrodes are provided between the back surface of the odor sensor 103 and the base material layer 153, and the electrodes on the base material layer 153 side are connected from the back of the electrode via a through hole or a via. Therefore, the electrodes can omit wiring, and further, the electrodes sandwich the wiring, so that the adhesion and desorption of the odor to and from the electrodes can be further suppressed. As described above, although Au is used as the first protective layer 152, the odor is not completely absorbed or desorbed thereto. Therefore, it is important to prevent the electrodes from being exposed as much as possible.

As described above, the odor sensor 103 itself is an element that requires electrical connection, and thus requires a circuit board. Therefore, efficient mounting can be achieved with a single printed circuit board by mounting the odor sensor 103 alone or only the odor sensor 103 and the IC on the front surface to reduce an unnecessary adsorption/desorption area, covering the other area with the first protective layer 152 to utilize the front surface as the sensor chamber 121 or one surface of the flow path, and using the back surface as a mounting surface for other circuit elements.

In particular, since the printed circuit board has a technique of covering Cu, Ni, and Au and the front surface with a noble metal, the first protective layer 152 can be easily formed by this technique.

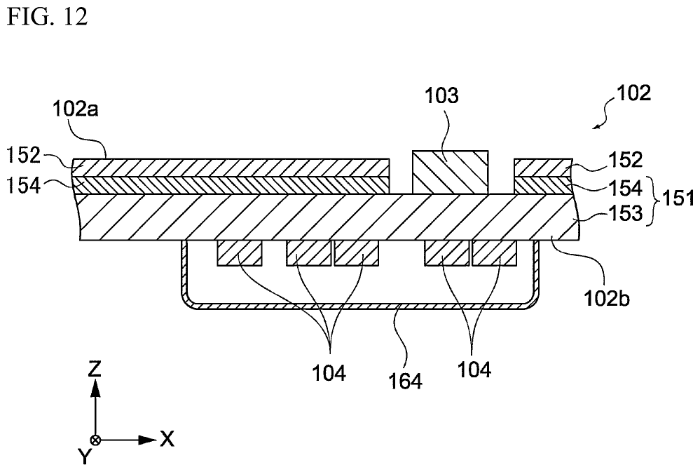

The electronic components 104 are mounted on the second main surface 102b, but may be shielded by a shield member. FIG. 12 is a cross-sectional view illustrating the electronic components 104 shielded by the shield member 164. The shield member 164 is made of metal and is bonded to the second main surface 102b to form an RF (Radio Frequency) shield. Since the odor sensor 103 is not mounted on the second main surface 102b, the second main surface 102b is covered with the shield member 164, and thus, noise can be reduced. This is particularly effective when the odor sensor 103 is a piezoelectric resonator type that operates in a high frequency band. Further, in the case that the first protective layer 152 is also grounded (GND) together with the shield member 164, substantially complete shielding can be achieved.

The housing member 101 has the inner surface 101c that forms the flow path F together with the flow path region 131 of the sensor substrate 102, and a second protective layer may be formed on the inner surface 101c.

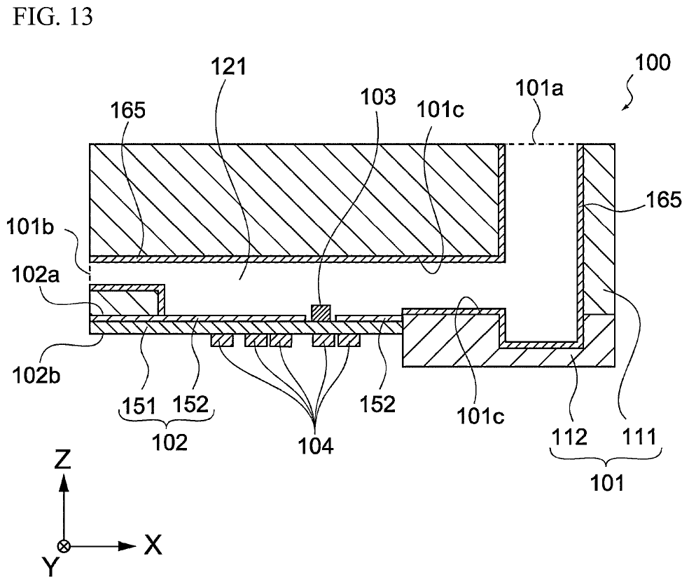

FIG. 13 illustrates the odor measuring device 100 provided with a second protective layer 165. As illustrated in FIG. 13, the second protective layer 165 is provided on the inner surface 101c. The second protective layer 165 is a layer that inhibits adhesion of odorous substances to the inner surface 101c. In particular, the second protective layer 165 reduces the surface energy of the inner surface 101c and inhibits the adhesion of odorous substances by reducing the specific surface area by planarization. The second protective layer 165 is made of amorphous silicon, diamond-like-carbon (DLC), a noble metal, or a composite layer thereof. The thickness of the second protective layer 165 is preferably 40 μm or greater and 200 μm or less.

Furthermore, the odor measuring device 100 may include a reference sensor.

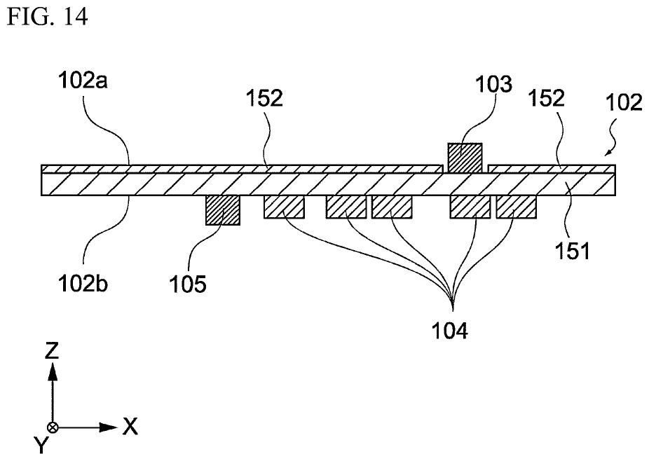

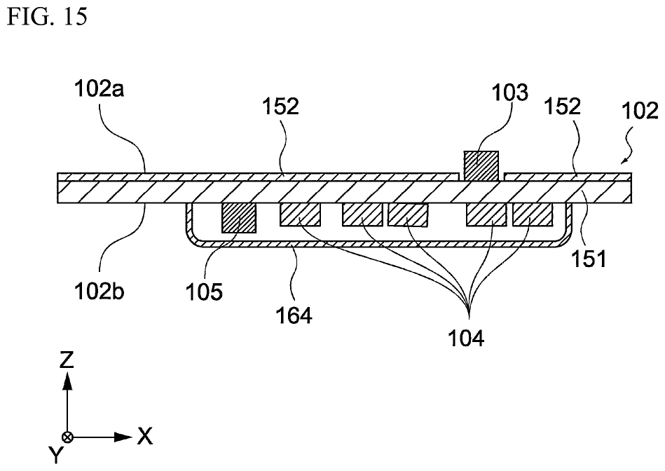

FIG. 14 and FIG. 15 illustrate the sensor substrate 102 on which a reference sensor 105 is mounted. As illustrated in FIG. 14 and FIG. 15, the reference sensor 105 is mounted on the second main surface 102b, and may be covered with a shield member 164 as illustrated in FIG. 15.

The reference sensor 105 includes an adsorption film made of the same material as the adsorption film of the odor sensor 103, and is used for temperature correction of the odor sensor 103. The odor sensor 103 with high sensitivity is highly sensitive to disturbance, and unintended noise or drift often occurs during sensing. Therefore, by mounting the reference sensor 105 on the second main surface 102b not facing the flow path F and eliminating the influence of the measurement target gas and humidity on the reference sensor 105, the same sensitivity to temperature as the odor sensor 103 is achieved, and the reference sensor 105 can be used for temperature correction.

### [Method of Manufacturing Odor Measuring Device]

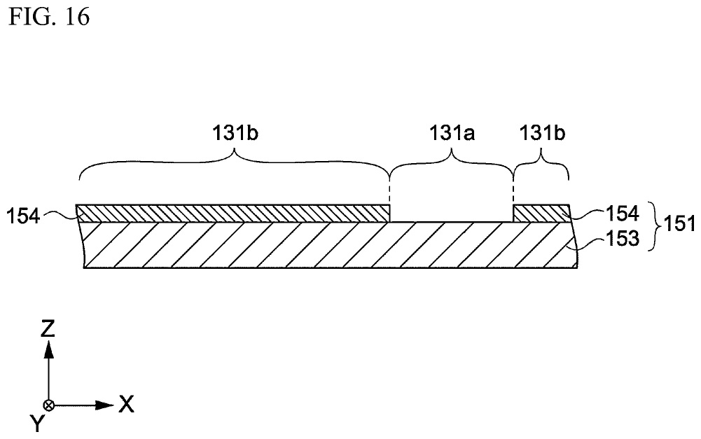

The odor measuring device 100 can be manufactured as follows. FIG. 16 to FIG. 18 are schematic views illustrating a method of manufacturing the odor measuring device 100. First, as illustrated in FIG. 16, the substrate body 151 is fabricated or prepared. The substrate body 151 is formed by stacking the metal layer 154 on the base material layer 153 in the non-mounting region 131b by plating or the like.

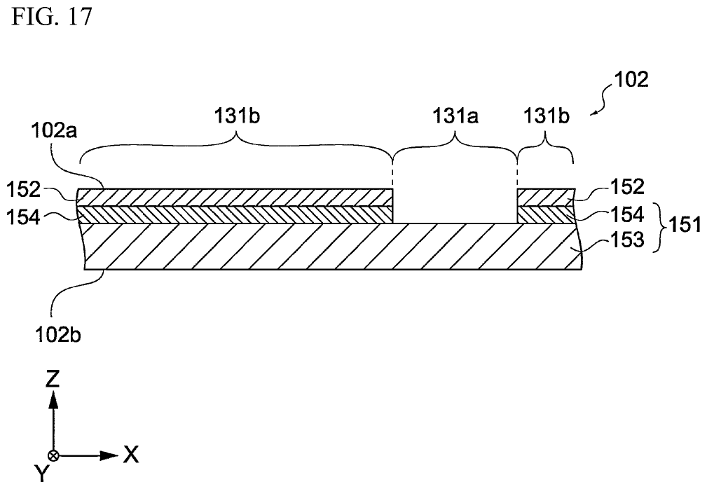

Next, as illustrated in FIG. 17, the first protective layer 152 is stacked on the metal layer (Cu/Ni layer) 154 in the non-mounting region 131b to fabricate the sensor substrate 102. The first protective layer 152 made of DLC or amorphous Si can be formed by masking the odor sensor mounting region 131a and performing a dry process such as chemical vapor deposition (CVD), sputtering, or vapor deposition.

On the other hand, when the first protective layer 152 is made of a noble metal such as Au or Pt, the electrodes of the odor sensor 103 are provided in the mounting region, and this portion is exposed from the mask and formed by plating or the like.

In the drawings, electrodes are omitted for simplification.

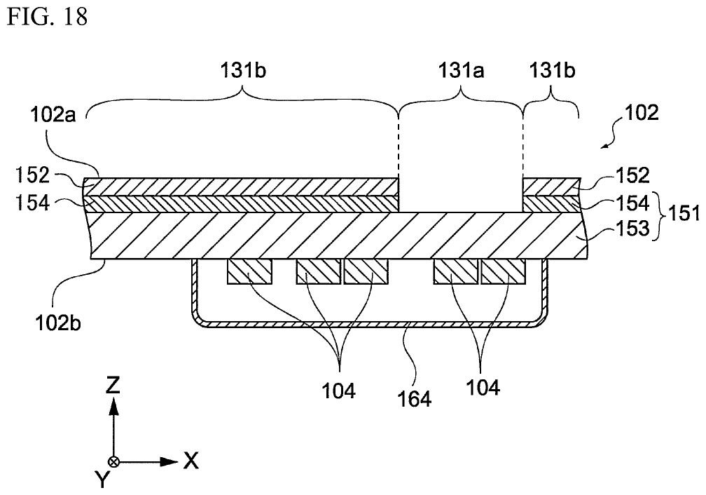

Then, as illustrated in FIG. 18, the electronic components 104 are mounted on the second main surface 102b, and the electronic components 104 are covered with the shield member 164 as necessary.

Then, as illustrated in FIG. 10 or FIG. 11, the odor sensor 103 is mounted in the odor sensor mounting region 131a.

Finally, as illustrated in FIG. 2, the sensor substrate 102 is joined to the first housing member 111 and the second housing member 112, thereby enabling hermetic sealing for protecting the sensor chamber 121 from the outside air. The sensor substrate 102 and the housing member 101 can be joined by screwing via a packing. When the housing member 101 is made of metal, the metal layer 154 may be exposed in the sensor substrate 102, and the housing member 101 and the metal layer 154 may be bonded at room temperature. The odor measuring device 100 can be manufactured as described above.

In this manufacturing method, when amorphous Si, DLC, or the like is formed by CVD or the like, the electronic component 104 and the odor sensor 103 are mounted on the sensor substrate 102 after the step of forming the first protective layer 152, and therefore, deterioration of each component due to the step of forming the first protective layer 152 can be avoided.

### [Effects of Odor Measuring Device]

In the odor measuring device 100, as indicated by arrows in FIG. 5, the measurement target gas flows through the flow path F between the intake port 101a and the exhaust port 101b. The odorous substance contained in the measurement target gas is adsorbed by the odor sensor 103, and the odor sensor 103 outputs a detection value corresponding to the adsorption amount of the odorous substance to the electronic component 104. The electronic component 104 performs various signal processing on the detection value.

As illustrated in FIG. 6, the first main surface 102a of the sensor substrate 102 has the flow path region 131 forming the flow path F, and the flow path region 131 is composed of the odor sensor mounting region 131a and the non-mounting region 131b. The odor sensor 103 is mounted in the odor sensor mounting region 131a, and the first protective layer 152 is provided in the non-mounting region 131b as illustrated in FIG. 8. Therefore, the first protective layer 152 inhibits adhesion of the odorous substance contained in the measurement target gas flowing through the flow path F to the sensor substrate 102, and the influence of adhesion and desorption on the detection value of the odor sensor 103 is prevented. Further, the electronic components 104 are mounted on the second main surface 102b, which does not face the flow path F, and the adhesion of the odorous substance to the electronic components 104 is also prevented. Furthermore, the electronic components 104 can be covered with the shield member 164, and the influence of noise can be reduced.

### [Mounting Surface of Electronic Components]

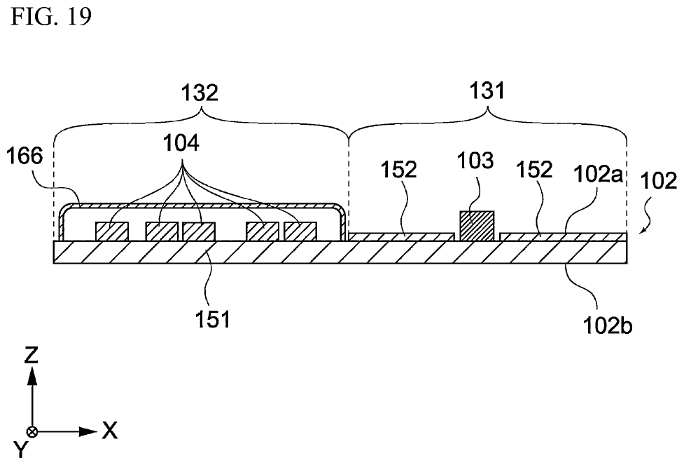

The electronic components 104 may be mounted on the first main surface 102a. FIG. 19 is a cross-sectional view of the sensor substrate 102 in which the electronic components 104 are mounted on the first main surface 102a. As illustrated in FIG. 19, the electronic components 104 are mounted in the non-flow path region 132 of the first main surface 102a and are sealed by a sealing member 166. The sealing member 166 is made of a metal or a resin, and is bonded to the first main surface 102a. The region of the first main surface 102a sealed by the sealing member 166 does not face the flow path F illustrated in FIG. 5, and is the non-flow path region 132. The odor sensor mounting region 131a of the flow path region 131 may also be covered by the odor sensor 103 and become the non-flow path region 132. When the sealing member 166 is made of a resin, a coating layer for preventing adhesion of an odorous substance may be provided on the surface of the sealing member 166. In this configuration, the adhesion of the odorous substance contained in the measurement target gas flowing through the flow path F to the sensor substrate 102 is also inhibited, and the influence of adhesion and desorption on the detection value of the odor sensor 103 is prevented.

### [Other Configurations of Sensor Substrate]

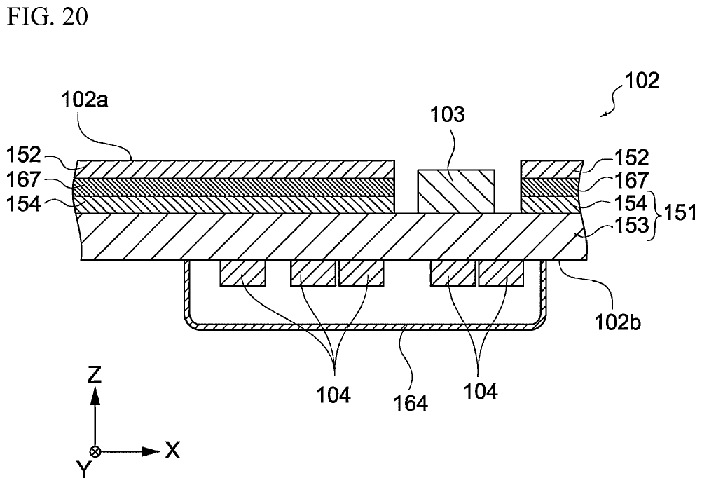

The first protective layer 152 may be formed on the substrate body 151 using a support. FIG. 20 is a cross-sectional view illustrating the sensor substrate 102 including a support 167. The support 167 is a metal plate or a metal foil, and is attached to the metal layer 154 with an adhesive material or a double-sided tape. The first protective layer 152 is stacked on the support 167.

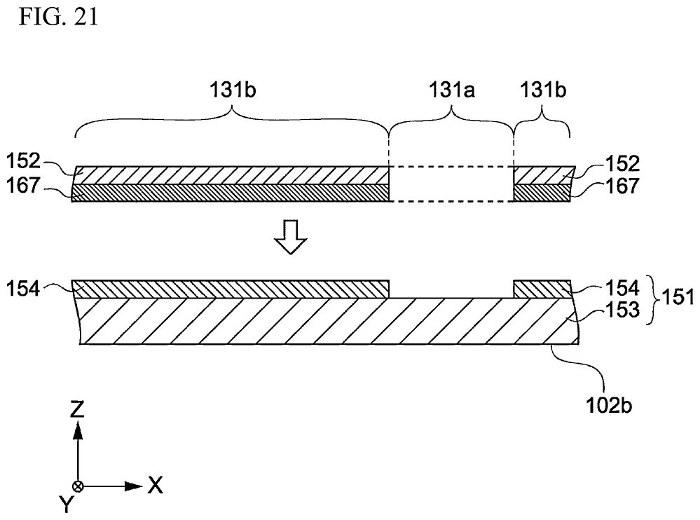

FIG. 21 is a schematic view illustrating a method of manufacturing the sensor substrate 102. As illustrated in FIG. 21, the first protective layer 152 is stacked on the support 167 in advance. The support 167 is hollowed out in the region corresponding to the odor sensor mounting region 131a. The sensor substrate 102 can also be manufactured by attaching the support 167 to the substrate body 151.

### [Arrangement of Flow Path Region]

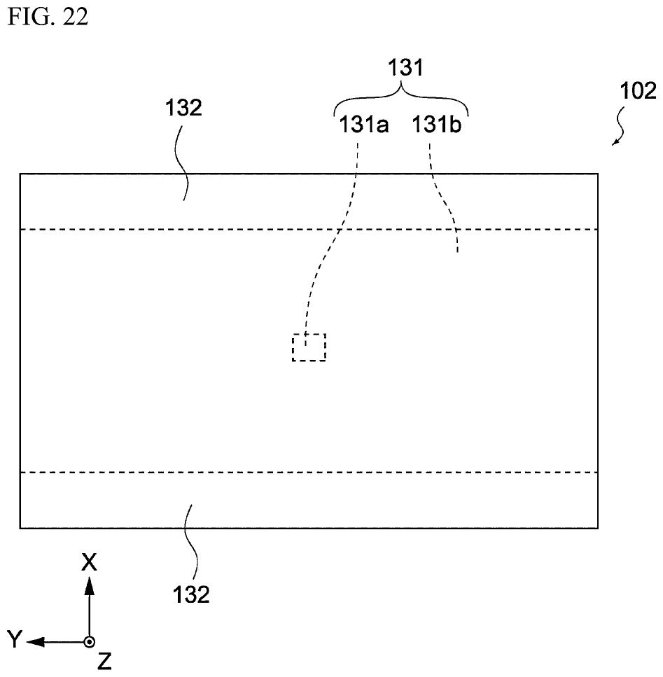

The flow path region 131 facing the flow path F is provided on the first main surface 102a of the sensor substrate 102 as described above. FIG. 22 to FIG. 24 are plan views illustrating arrangement examples of the flow path region 131 on the first main surface 102a. As illustrated in FIG. 22, the non-flow path regions 132 may be provided along the long side of the sensor substrate 102, and the flow path region 131 may be provided between the non-flow path regions 132.

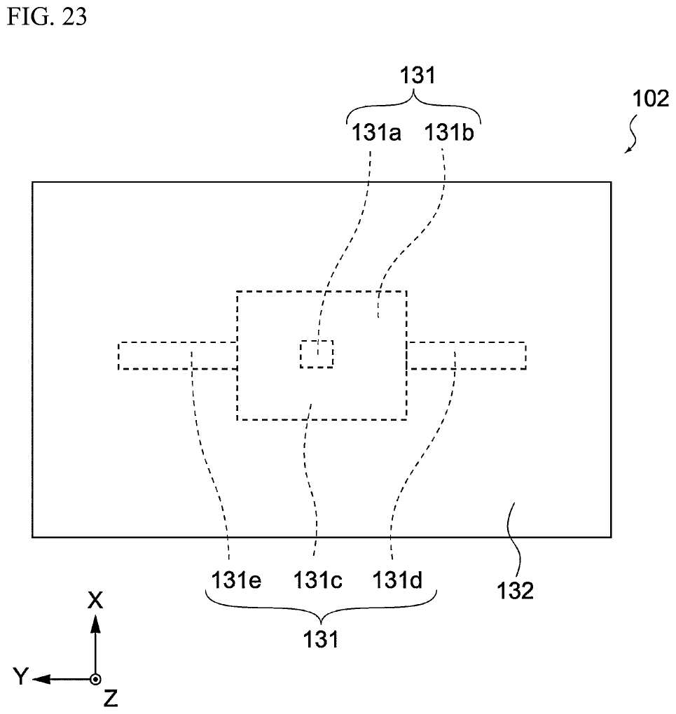

As illustrated in FIG. 23, the flow path region 131 may be constituted by a sensor chamber region 131c, an intake flow path region 131d, and an exhaust flow path region 131e. The sensor chamber region 131c is a region constituting the sensor chamber 121 illustrated in FIG. 3, and includes the odor sensor mounting region 131a. The intake flow path region 131d is a region constituting an intake flow path through which the measurement target gas is sucked into the sensor chamber 121, and is formed by a groove provided in the housing member 101. The exhaust flow path region 131e is a region constituting an exhaust flow path through which the measurement target gas is discharged from the sensor chamber 121, and is formed by a groove provided in the housing member 101. The non-mounting region 131b is a region other than the odor sensor mounting region 131a among the sensor chamber region 131c, the intake flow path region 131d, and the exhaust flow path region 131e.

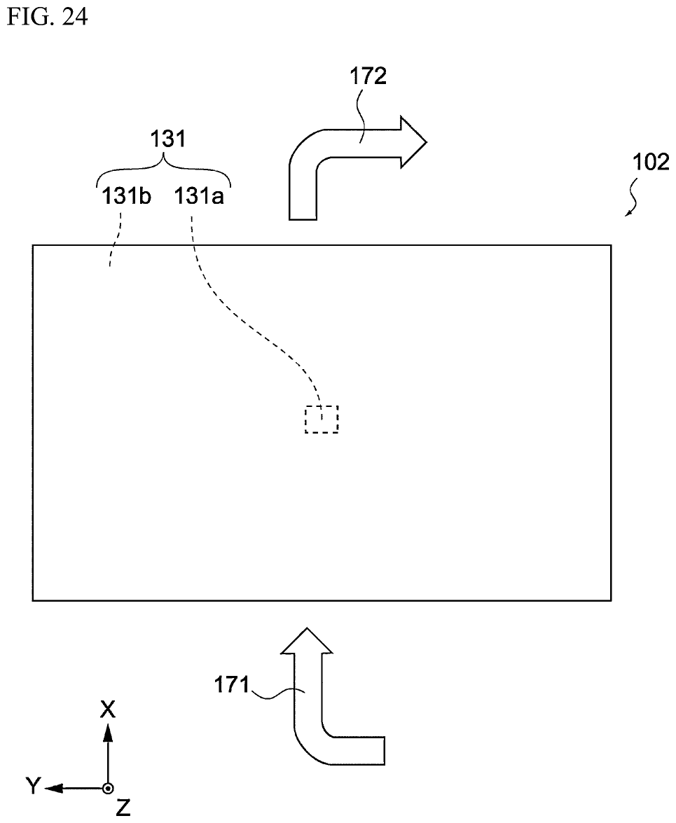

Further, as illustrated in FIG. 24, the entire region of the first main surface 102a may be the flow path region 131. The measurement target gas flows into the sensor chamber 121 formed by the flow path region 131 via an intake flow path 171, and is discharged from an exhaust flow path 172. The intake flow path 171 and the exhaust flow path 172 are, for example, pipes.

### [Implementation of Plurality of Odor Sensors]

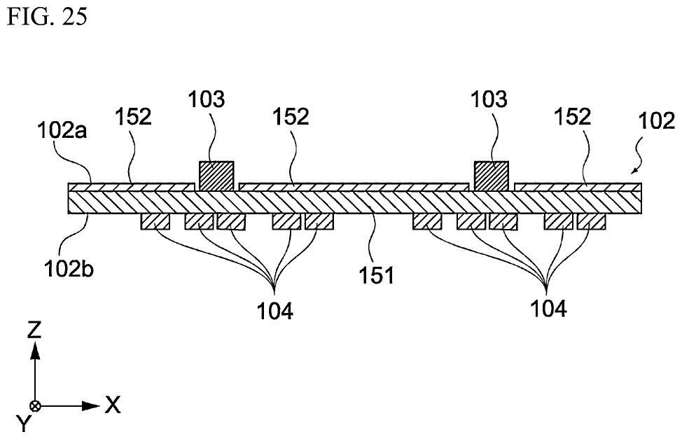

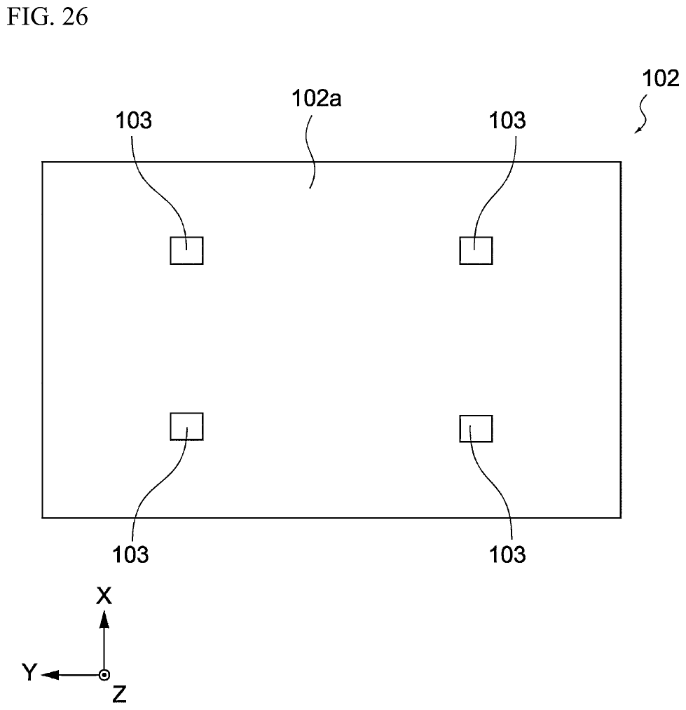

In the odor measuring device 100, the odor can be identified by using a plurality of the odor sensors 103 having different types of adsorption films as described above. Hereinafter, the structure of the odor measuring device 100 including the odor sensors 103 of four channels will be described as an example. FIG. 25 is a cross-sectional view of the sensor substrate 102 on which the odor sensors 103 of four channels are mounted, and FIG. 26 is a plan view of the sensor substrate 102.

As illustrated in FIG. 25 and FIG. 26, when four odor sensors 103 are mounted on one sensor substrate 102, the sensor substrate 102 has no seam, allowing the measurement target gas to efficiently flow. On the other hand, if any one of the odor sensors 103 has a characteristic defect, all of the four odor sensors 103 cannot be used, and thus the yield may be reduced.

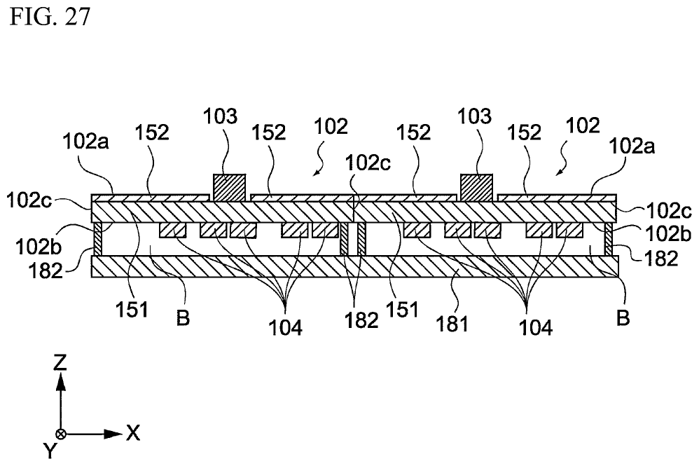

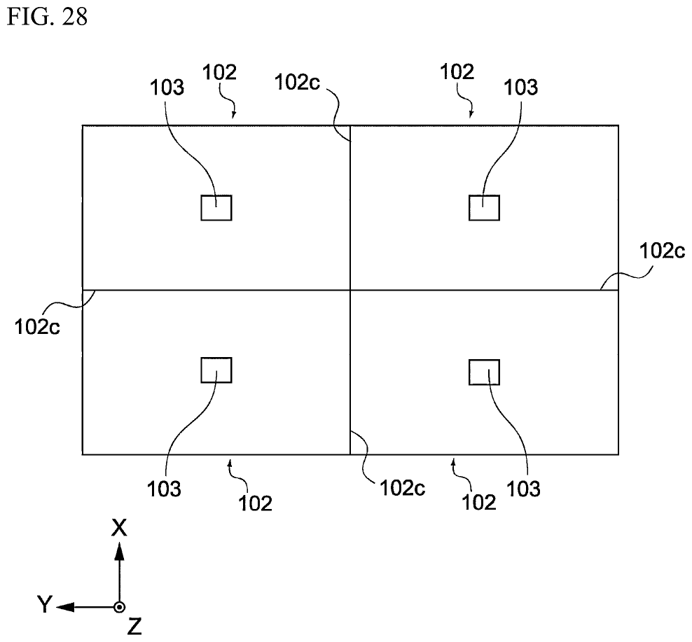

Therefore, in the odor measuring device 100, a plurality of the sensor substrates 102 may be implemented by using a support substrate. FIG. 27 is a cross-sectional view illustrating a support substrate 181 and a plurality of the sensor substrates 102, and FIG. 28 is a plan view of the sensor substrates 102 mounted on the support substrate 181. As illustrated in FIG. 27 and FIG. 28, the sensor substrates 102 are disposed in a direction in which the second main surface 102b faces the support substrate 181, and are supported by the support substrate 181 by pins 182 and electrically connected. As illustrated in FIG. 27, when the end surface of each sensor substrate 102 is defined as an end surface 102c, the sensor substrates 102 are arranged so that the end surfaces 102c thereof are in contact with each other, and a space is formed between the second main surface 102b and the support substrate 181.

Hereinafter, a space between the second main surface 102b and the support substrate 181 is referred to as a back surface space B. The electronic components 104 are mounted on the second main surface 102b, and the electronic components 104 are located in the back surface space B. Each sensor substrate 102 may be connected to the support substrate 181 by a connector instead of the pins 182.

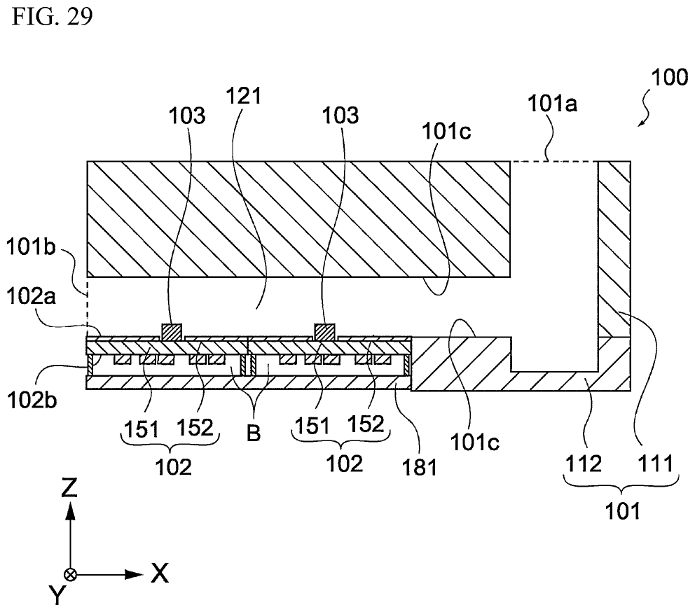

FIG. 29 is a cross-sectional view illustrating the housing member 101, the sensor substrate 102, and the support substrate 181. As illustrated in FIG. 29, the sensor substrates 102 are disposed so that the first main surfaces 102a are on the sensor chamber 121 side, and the back surface spaces B are separated from the sensor chamber 121 by the sensor substrates 102.

In this structure, even when any of the odor sensors 103 has a characteristic defect, the sensor substrate 102 can be replaced as a whole, which is advantageous in terms of cost. In addition, when the odorous substance to be measured is changed, the combination of the odor sensors 103 can be easily changed. On the other hand, if there is a gap between the sensor substrates 102, the measurement target gas may flow into the back surface space B through the gap between the sensor substrates 102, and the odorous substance may adhere to the electronic components 104, the support substrate 181, and the like. Conversely, there is a possibility that gas from the back surface space B enters the sensor chamber.

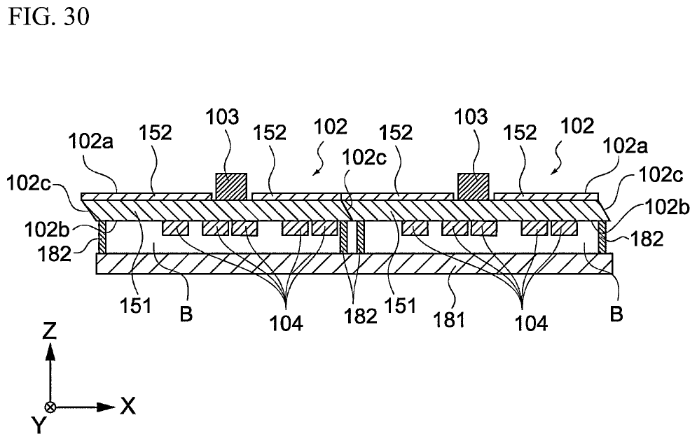

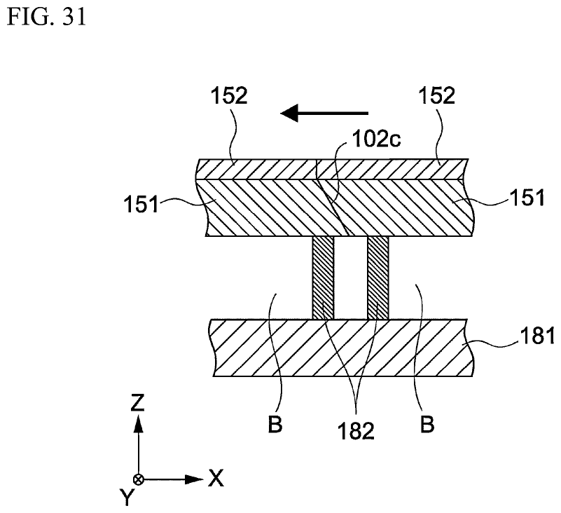

Therefore, a taper may be provided on the end surface 102c of each sensor substrate 102. FIG. 30 is a cross-sectional view illustrating the sensor substrate 102 and the support substrate 181 in which the end surface 102c is tapered, and FIG. 31 is an enlarged view of FIG. 30.

As illustrated in FIG. 31, the end surface 102c of the sensor substrate 102 is provided with a taper that fits with the end surface 102c of the adjacent sensor substrate 102. In FIG. 31, when the measurement target gas flows in the direction indicated by the arrow, the measurement target gas is less likely to flow into the back surface space B by providing a taper inclined in the direction opposite to the direction in which the measurement target gas flows as illustrated in FIG. 31.

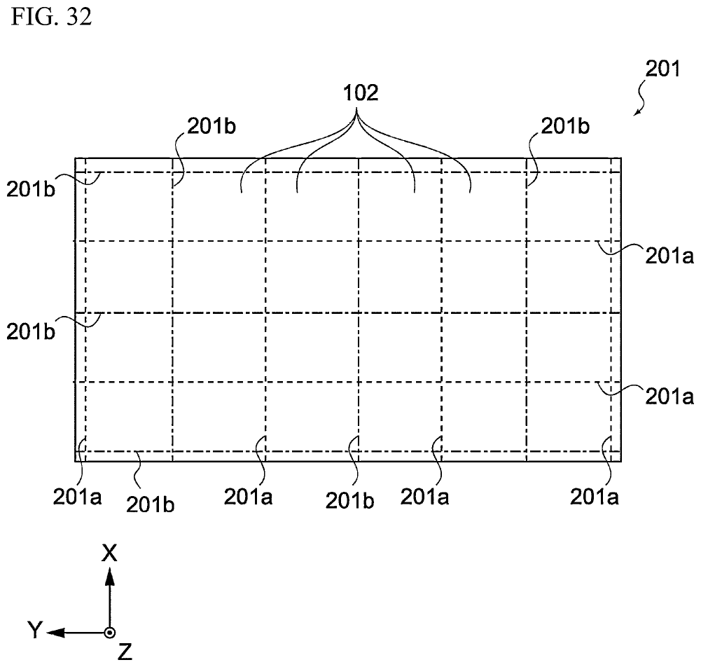

FIG. 32 is a schematic diagram illustrating a method of forming the sensor substrate 102 in which such a taper is provided on the end surface 102c.

As illustrated in FIG. 32, a sensor substrate 201 including a plurality of the sensor substrates 102 is formed, and V-shaped grooves 201a are formed in a lattice shape on the front surface and V-shaped grooves 201b are formed in a lattice shape on the back surface. By dividing the sensor substrate 201 using the V-shaped grooves 201a and the V-shaped grooves 201b, a plurality of the sensor substrates 102 each having the tapered end surface 102c can be formed. The first protective layer 152 and the odor sensor 103 may be provided on the sensor substrate 201 in advance, or may be provided after the sensor substrate 201 is divided.

### [Electrical Connection of Sensor Substrate]

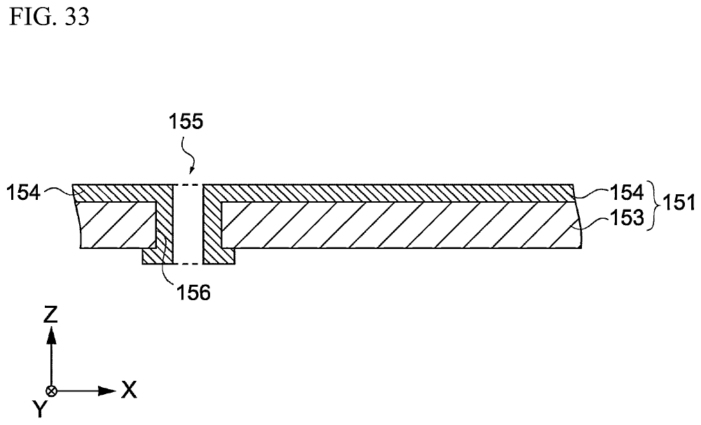

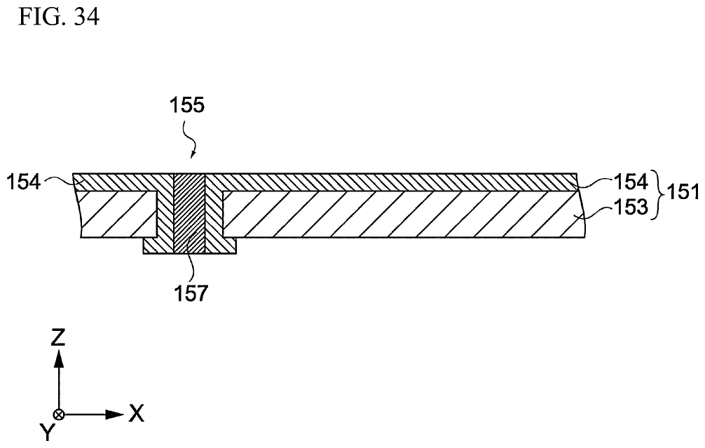

The electrical connection of the sensor substrate 102 will be described. FIG. 33 and FIG. 34 are cross-sectional views of the substrate body 151.

As illustrated in FIG. 33, a through hole 155 (or via) is provided in the substrate body 151. The through hole 155 is formed of a through hole 156 provided in the base material layer 153 and the metal layer 154 covering the inner surface of the through hole 156, and is used for electrical connection between the first main surface 102a and the second main surface 102b.

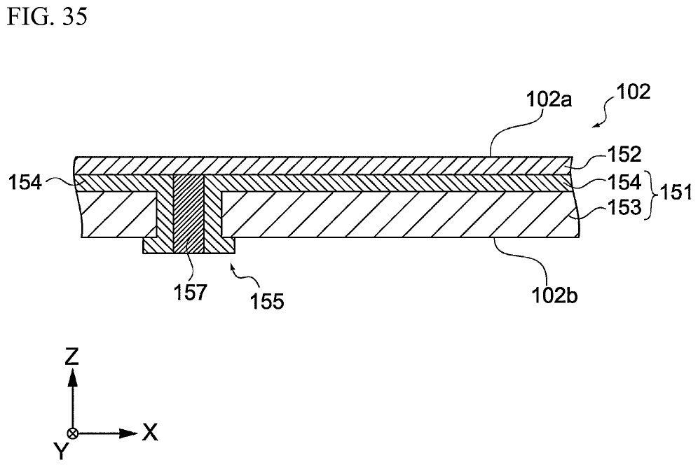

Here, as illustrated in FIG. 34, the through hole 155 located in the flow path region 131 is preferably filled with a plug 157. By filling the through hole 155, the odorous substance can be prevented from adhering to the inside of the through hole 155 or the second main surface 102b. The plug 157 is made of resin, metal plating, solder, conductive paste, or the like, and is not particularly limited. FIG. 35 is a cross-sectional view of the sensor substrate 102 including the substrate body 151 having the through holes 155 and the first protective layer 152. As illustrated in FIG. 35, the through holes 155 are covered with the first protective layer 152.

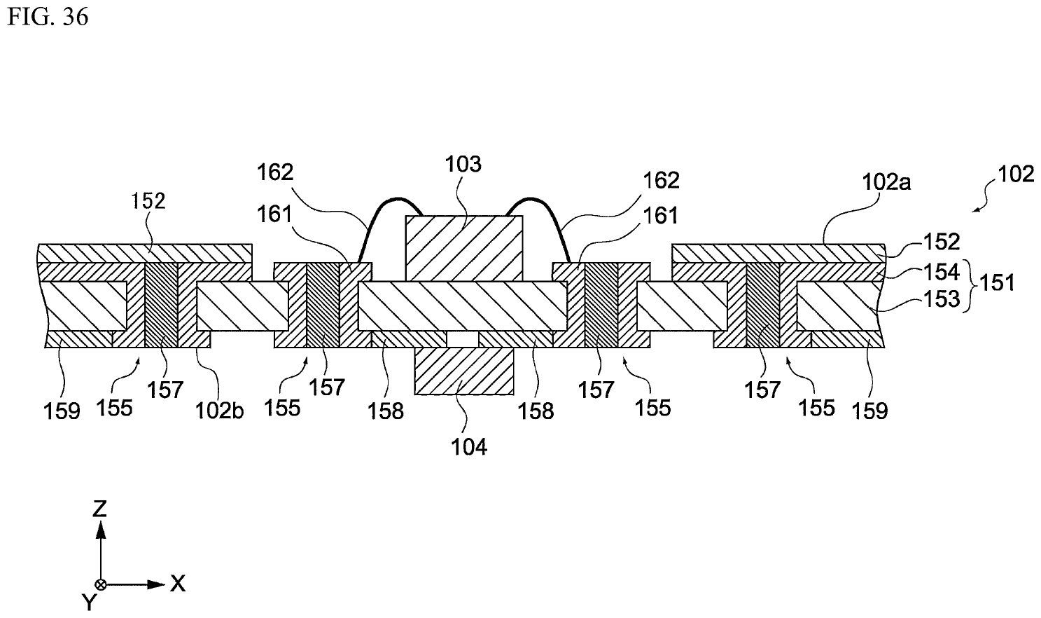

FIG. 36 is a schematic view illustrating electrical connection of the sensor substrate 102, the odor sensor 103, and the electronic component 104 by the through hole 155.

As illustrated in FIG. 36, first conductive patterns 158 connected to the electrodes 161 via the through holes 155 are provided on the second main surface 102b. The electronic component 104 is mounted on the second main surface 102b and is electrically connected to the odor sensor 103 via the first conductive pattern 158. Further, a second conductive pattern 159 connected to a ground is provided on the second main surface 102b, and the metal layer 154 is electrically connected to the second conductive pattern 159 via the through hole 155.

Further, a cover forming the sensor chamber 121 may be joined to the sensor substrate 102 instead of the housing member 101.

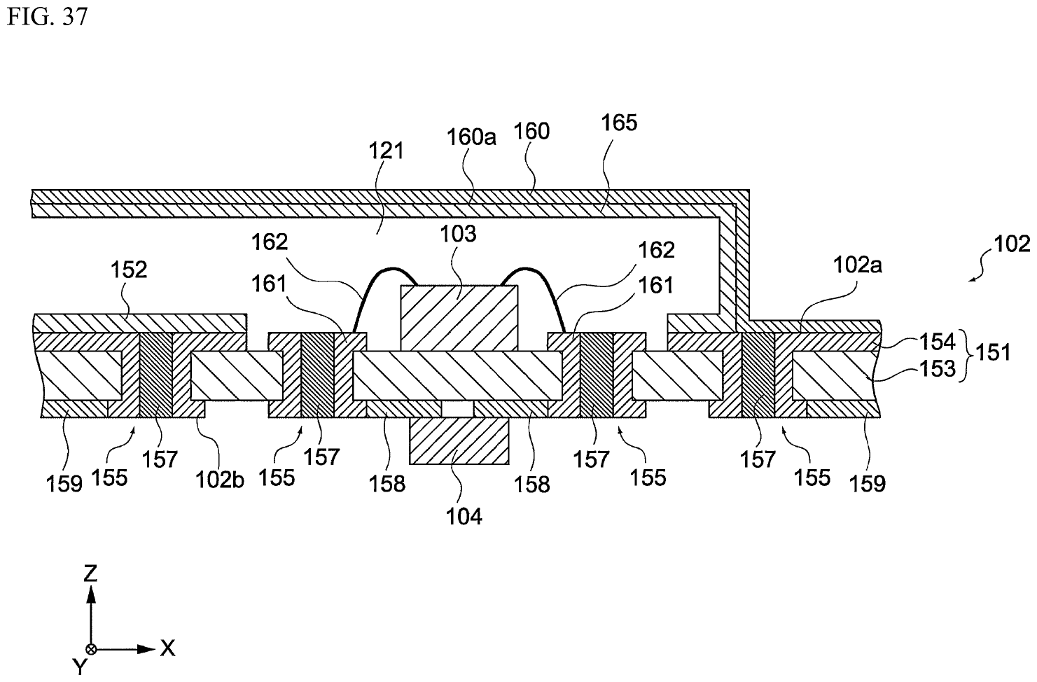

FIG. 37 is a cross-sectional view illustrating the sensor substrate 102 to which a cover 160 is bonded.

As illustrated in FIG. 37, the second protective layer 165 for inhibiting adhesion of odorous substances to an inner surface 160a is provided on the inner surface 160a of the cover 160. The cover 160 is made of a metal such as stainless steel, and is bonded to the metal layer 154 and is grounded via the metal layer 154. The second protective layer 165 is formed by surface-treating oxides on the metal surface constituting the cover 160 and smoothing the inner surface 160a to inhibit the adhesion of odorous substances.

### [Other Configurations of Flow Path]

In the above description, the flow path F is formed by the sensor chamber 121, but the flow path F may have the following configuration.

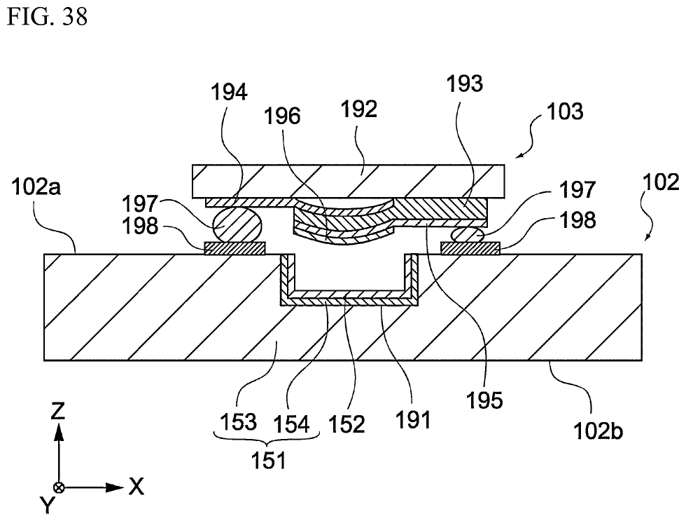

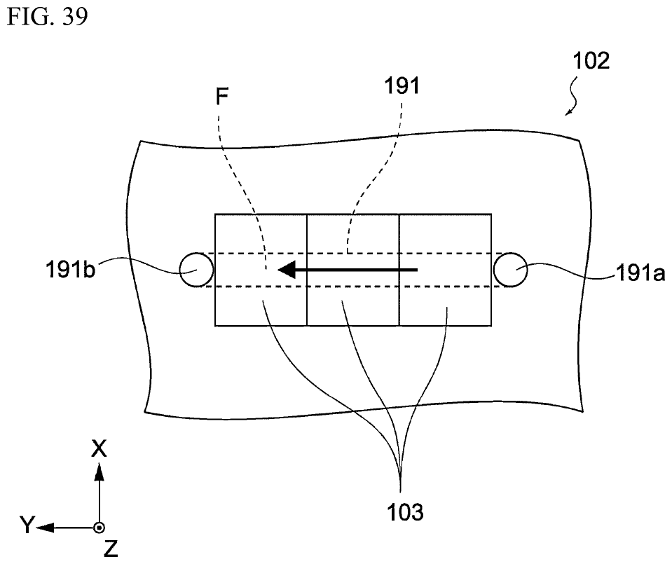

FIG. 38 is a cross-sectional view of the sensor substrate 102 in which a groove 191 is provided on the first main surface 102a, and FIG. 39 is a plan view of the sensor substrate 102. As illustrated in FIG. 38 and FIG. 39, the groove 191 extending in one direction is provided on the first main surface 102a. An intake port 191a is provided at one end of the groove 191, and an exhaust port 191b is provided at the other end. A gas delivery mechanism (not illustrated) is connected to the intake port 191a, and the measurement target gas flows from the intake port 191a through the groove 191 and is exhausted from the exhaust port 191b as indicated by the arrows in FIG. 39. That is, the groove 191 constitutes the flow path F, and the inner surface of the groove 191 corresponds to the flow path region 131.

The odor sensors 103 are mounted at both sides of the groove 191, and adsorb odorous substances contained in the measurement target gas flowing through the groove 191. Specifically, the odor sensor 103 includes an element substrate 192, a piezoelectric film 193, a lower electrode 194, an upper electrode 195, and an adsorption film 196, and the adsorption film 196 is bonded to electrodes 198 by bonding portions 197 in a direction in which the adsorption film 196 faces the groove 191, resulting in face-down flip-chip mounting. In the flip-chip mounting, in the case of solder reflow, there is a high possibility that a gap is formed between the sensor substrate 102 and the odor sensor 103, and therefore, the bonding between the bonding portion 197 and the electrode 198 is preferably performed by room-temperature bonding mounting of the same kind of metal such as Au—Au.

In this configuration, as illustrated in FIG. 38, the metal layer 154 and the first protective layer 152 are provided on the inner surface of the groove 191. In this configuration, the odorous substance contained in the measurement target gas flowing through the flow path F is also inhibited from adhering to the sensor substrate 102, and the influence of the adhesion and desorption on the detection value of the odor sensor 103 is prevented. The electronic components 104 can be mounted on either the first main surface 102a or the second main surface 102b.

### [Fluid]

In the present embodiment, the flow path F is a flow path through which gas flows, but the flow path F may be a flow path through which a fluid other than gas, such as liquid, flows. The odor sensor 103 can also detect an odorous substance contained in the fluid flowing through the flow path F.

Although the embodiment of the present invention has been described in detail above, the present invention is not limited to the specific embodiment, and various modifications and changes can be made within the scope of the gist of the present invention described in the claims.

What is claimed is:

1. An odor measuring device comprising:
a sensor substrate having a first main surface having a flow path region constituting a flow path, the flow path region including a first region and a second region;
an odor sensor that is mounted on the first region and detects an odorous substance; and
a first protective layer provided on the second region.

2. The odor measuring device according to claim 1, wherein the sensor substrate includes a base material layer, a metal layer stacked on the base material layer, and the first protective layer stacked on the metal layer.

3. The odor measuring device according to claim 1,
wherein the sensor substrate has a second main surface opposite to the first main surface, and
wherein the odor measuring device further comprises an electronic component mounted on a surface of the sensor substrate other than the first main surface and electrically connected to the odor sensor.

4. The odor measuring device according to claim 1,
wherein the first main surface further includes a non-flow path region that does not constitute the flow path, and
wherein the odor measuring device further comprises an electronic component that is mounted in the non-flow path region and electrically connected to the odor sensor.

5. The odor measuring device according to claim 4, further comprising a sealing member that is bonded to the non-flow path region and seals the electronic component.

6. The odor measuring device according to claim 1, wherein the sensor substrate includes a base material layer, a metal layer stacked on the base material layer, a support body that is a metal plate or a metal foil and is attached to the metal layer, and the first protective layer stacked on the support body.

7. The odor measuring device according to claim 1, further comprising a housing member that is joined to the sensor substrate, has an inner surface that forms the flow path together with the flow path region, and has a second protective layer provided on the inner surface, the second protective layer inhibiting adhesion of an odorous substance contained in a gas.

8. The odor measuring device according to claim 7, wherein the first protective layer and the second protective layer are made of the same material.

9. The odor measuring device according to claim 1, further comprising:
a support substrate,
wherein the sensor substrate is provided in plural,
wherein each of the sensor substrates have a second main surface opposite to the first main surface,
wherein each of the sensor substrates is connected to the support substrate,
wherein the second main surfaces of the sensor substrates face the support substrate, and
wherein the odor measuring device further comprises an electronic component that is mounted on the second main surface and electrically connected to the odor sensor.

10. The odor measuring device according to claim 9, wherein end surfaces of the sensor substrates are provided with a taper that fits with an end surface of an adjacent sensor substrate.

11. The odor measuring device according to claim 1,
wherein the sensor substrate has a second main surface opposite to the first main surface,
wherein the odor sensor includes a first adsorption film to which the odorous substance adsorbs, and
wherein the odor measuring device further comprises a reference sensor that is mounted on the second main surface and has a second adsorption film made of the same material as the first adsorption film.

12. An odor measuring device comprising:
a sensor substrate that has a first main surface and a second main surface, the first main surface having a first region and a second region, the first region including a sensor region to which an odor sensor for detecting an odorous substance is bonded and an electrode region that is adjacent to the sensor region and provided with an electrode to which the odor sensor is connected, the second main surface being a main surface opposite to the first main surface;
a first conductive pattern that is provided on the second main surface and is electrically connected to the electrode;
a metal layer that is provided in the second region;
a first protective layer that is stacked on the metal layer and inhibits adhesion of an odorous substance;
an odor sensor mounted on the first region and electrically connected to the electrode;
an electronic component mounted on the second main surface and electrically connected to the first conductive pattern; and
a sensor chamber having the first main surface as an inner surface.

13. The odor measuring device according to claim 12,
wherein the sensor substrate is provided with a second conductive pattern connected to a ground on the second main surface, and
wherein the metal layer is electrically connected to the second conductive pattern via a through hole provided in the sensor substrate.

14. The odor measuring device according to claim 12, wherein a flow path extending from the sensor chamber to the outside is provided on the first main surface, and a second protective layer that inhibits adhesion of an odorous substance constitutes an inner surface of the flow path.

15. An odor measuring device comprising:
a sensor substrate that has a first main surface including a flow path region constituting a flow path and includes a first protective layer provided in the flow path region, the first protective layer inhibiting adhesion of an odorous substance contained in a gas flowing through the flow path; and
an odor sensor that is mounted in the odor sensor mounting region and detects an odorous substance contained in the gas.

16. The odor measuring device according to claim 1, wherein the first protective layer is made of amorphous silicon, diamond-like-carbon (DLC), a noble metal, or a composite layer thereof.
